(19) United States
(12) Patent Application Publication
(10) Pub. No.: US 2011/0015584 A1
(43) Pub. Date: Jan. 20, 2011

(54) EASY-PASTING EXERCISE PATCH

(76) Inventor: YUNG-FA SU, Yen Chao Hsiang (TW)

Correspondence Address: LEONG C. LEI, PMB # 1008, 1839 YGNACIO VALLEY ROAD, WALNUT CREEK, CA, 94598, US

(21) Appl. No.: 12/687,876

(22) Filed: Jan. 15, 2010

(30) Foreign Application Priority Data
Jul. 16, 2009 (TW) 098212950

(51) Int. Cl. A61M 35/00

(52) U.S. Cl. 604/289

(57) ABSTRACT

An easy-pasting exercise patch includes a patch body having an under surface to which a release paper is attached. The patch body is in the form of a thin sheet having a bottom having a center serving as a reference to define a plurality of turns of non-adhesive-coated circumferential slots in the form of concentric circles. The patch allows ready application by a user to his or her waist or abdomen. After the patch has been pasted, due to the arrangement of the circumferential slots, a radiating stretching force is induced toward the center to force the muscles of the pasted site to do autonomous wriggles for realizing the effect of exercise and removing excessive fat from the pasted site of the human body.

### (a) TECHNICAL FIELD OF THE INVENTION

The present invention generally relates to an easy-pasting exercise patch, and more particularly to a patch that allows ready application by a user to the abdomen to remove fat from the abdomen.

### (b) DESCRIPTION OF THE PRIOR ART

Health is the most important wealth of a person. Excellent health allows a person to participate in various activities of work and daily living. With the change of human society from agricultural civilization to industrialized civilization, the economic power of individuals is greatly improved, so that the modern people may significantly improve their living. Due to the economic prosperity, modern people consume more food but take less exercise, leading to excessive fat and cellulite of human body. To improve such a problem, besides change of the way of nutrition, successive and proper exercise is also an important issue. However, most people are not persistent to take exercise everyday for a long period of time. Some people take the solution of consuming less amount of food, but this leads to health problem due to insufficient supply of nutrition. The other people take medicines to lose weight, by which diarrhea may be caused to lose weight. However, this way may lead to impairment of body health and may soon gain the weight back after stopping taking the medicine. This is apparently not a proper way of losing weight. Experiments have been taken on some twenty persons of different ages by pasting exercise tapes, which are used to warp around a predetermined site of a human body to provide the effect of inflammation reduction and exercise damage protection, on the abdomen in a concentric circular manner. The size of waist is reduced by six centimeters in at most two months with such an application of the tape. The reason for the effect is that the exercise tape possesses excellent resiliency and stretching strength, so that when it is applied in a concentric circular manner to the human body, due to the stretching of the tape in applying the tape, the resiliency and the restoring stretching force of the exercise tape after being applied caused the muscles of human body at the application site to take forced motion by the memorization capability thereof for removing fat. This way of applying the exercise tape to the human body is a moderate manner of removing fat that does not cause harm to human body. However, this way is disadvantageous in that to apply the exercise tape in a concentric circular fashion with the belly button as the center to a user's abdomen by the user himself or herself is a difficult job. Further, since the exercise tape is resilient and possesses stretching strength, if the application of force is uneven during the process of applying the concentric circles of the tape, the tape so applied may show excessively tight or excessively loose sections, which lead to improper and non-uniform stretching force applied to the human body and eventually worsening the result of fat removal. Further, shrinkable abdomen belt is available in the market and such a band only squeeze the user's body and does not provide a centralizing effect.

### SUMMARY OF THE INVENTION

The primary objective of the present invention is to provide an easy-pasting exercise patch, and in particular an exercise patch that allows ready application by a user and may maintain proper and uniform tightness.

Another objective of the present invention is to provide an easy-pasting exercise patch, and in particular to a stretchable patch, which, after being applied to a predetermined site of human body, functions to remove fat from the site of the human body.

The easy-pasting exercise patch of the present invention possesses excellent resiliency and stretching strength, comprising a patch body having an under surface to which a release paper is attached. The patch body is in the faun of a thin sheet having bottom of which a center serves as a reference to define a plurality of non-adhesive-coated concentric circular circumferential slots. In practical embodiments, the circumferential slots can be set in the form of a plurality of ring-shaped and intercepted slots, or a helically-shaped slot. This allows a user to readily apply the patch to a desired site on the abdomen and a uniform stretching force is induced in the pasted patch. The patch, after being pasted, induces radiating stretching force toward a center with the non-adhesive-coated circumferential slots thereof, which, together with the memorization capability of muscles, forces successive wriggle of the muscles of the pasted site to thereby induce 360-degree uniform and centralized concentration toward the center, leading to an autonomous motion, which removes excessive fats of the pasted site.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is an exploded view of a patch constructed in accordance with a preferred embodiment of the present invention.

FIG. 2 is a top plan view of FIG. 1.

FIG. 3 is a top plan view of a patch constructed in accordance with another embodiment of the present invention.

FIGS. 4 and 5 demonstrate the application of the patch of the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

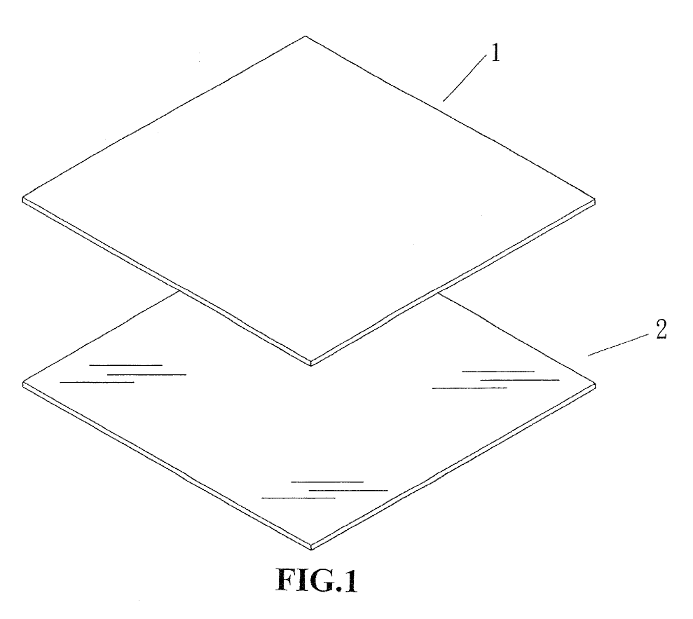

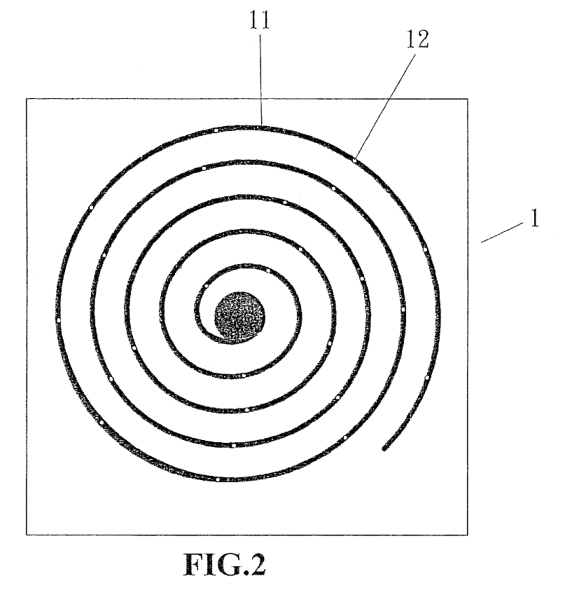

Referring first to FIGS. 1 and 2, an exercise patch according to the present invention comprises a patch body 1 having an under surface to which a release paper 2 is attached. The patch body 1 is of excellent resiliency and stretchability and is set in the form of a thin sheet of a predetermined shape having a bottom having a center. The patch body 1 forms a plurality of turns of non-adhesive-coated helically-shaped slot 11 extending in a circumferential direction in the form of concentric circles with the center of the bottom as a reference point. Further, in practice, a plurality of spaced air holes 12 may be defined in the helical slot 11.

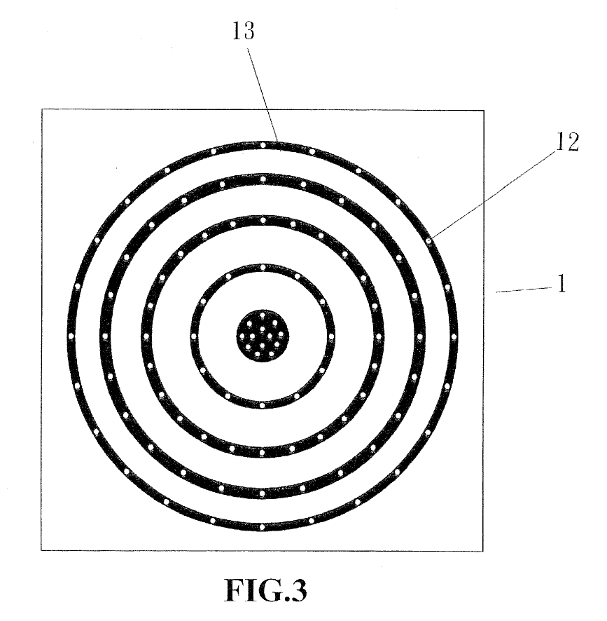

Also referring to FIG. 3, a bottom view of another embodiment of an exercise patch in accordance with the present invention is shown, wherein the exercise patch comprises a patch body 1 having a center serving as a reference point to define a plurality of turns of circumferentially extending ring-shaped non-adhesive-coated slots 13 of various sizes. In practice, air holes 14 may be defined in the slots 13.

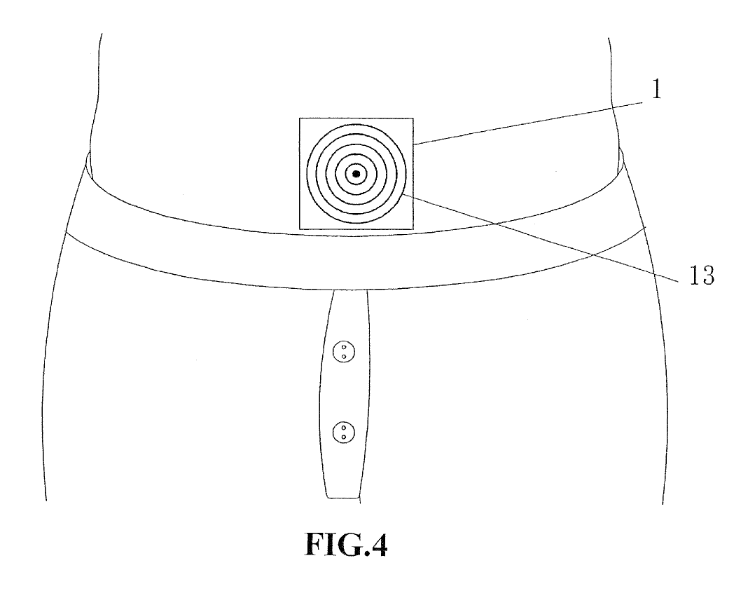

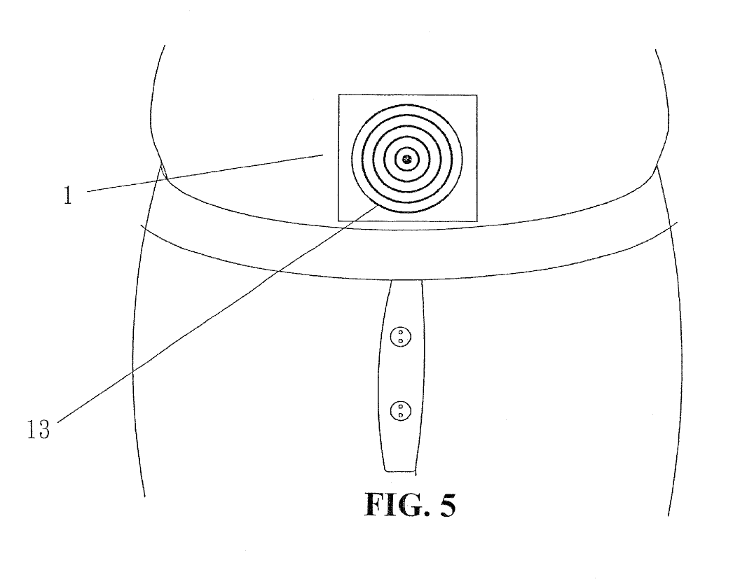

Referring to FIGS. 4 and 5, to apply the patch of the present invention to the abdomen of a user, the user first removes the release paper 2 from the under surface of the patch body 1 and then the user strains his or her muscles to shrink the abdomen. And then, the user applies the patch by regularly pasting the patch body 1 to a desired site of the body. Afterwards, the user relaxes the muscles to release the abdomen. The user is allowed to readily apply the patch to a predetermined site on the waist or the abdomen without the assistance of the other people. This simplifies the operation of application of the patch. Further, after the application of the patch, although the release of the strained or shrunk abdomen may lead to expansion of the site where the patch is pasted, the patch is of sufficient resilience and stretching strength, the patch body 1 can expand with the relaxation of the abdomen. Due to the resilience and stretching strength of the patch itself, the patch body 1 is forced to restore the original shape thereof. This, working with the memorization capability of the muscles of the site where the patch is applied, induces a radiating stretching force toward the center and forms 360-degree uniform concentration toward the center of the pasted site, causing repeated and successive wriggle of the muscles at that site. In other words, the muscles are set in successive wriggle, which leads to successive autonomous motion of the muscles of the site, by which fat of human body is eliminated and removed. The structure of the present invention allows for ready and easy application by a user himself or herself and during the period of use, since no medicine is used, and instead, the patch body 1 and the memorization capability of the muscles are used to provide the desired effect, no damage to the human body may occur. Further, the user does not need to take a great amount of exercise and only control of nutrition and food may realize the desired effect. Further, the air holes 12 provide better ventilation for the skin of the pasted site.

The patch body 1 can be coated with medicines of therapeutic effects of refreshing, inflammation-reducing, and pain-relieving, so that when the patch is applied to the waist or abdomen, besides the excitement of autonomous motion of muscles for fat removal induced in the applied site by the patch, the medicines coated on the patch also realizes the therapeutic effects pursued.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A patch comprising a patch body having an under surface to which a release paper is attached, wherein the patch body comprises a thin sheet having a bottom having a center serving as a reference to define at least a plurality of turns of non-adhesive-coated circumferential slots in the form of concentric circles.

2. The patch according to claim 1, wherein the circumferential slots are set in the form of a helical shape or an intercepted ring shape.

3. The patch according to claim 1, wherein the under surface of the patch body is coated with medicine.

4. The patch according to claim 1, wherein a plurality of spaced air holes is defined in the slot.
